(19) United States
(12) Patent Application Publication
(10) Pub. No.: US 2023/0102078 A1
(43) Pub. Date: Mar. 30, 2023

(54) LIGHT-EMITTING PARTICLES

(71) Applicant: Sumitomo Chemical Co., Ltd., Tokyo (JP)

(72) Inventors: Jonathan Behrendt, Godmanchester (GB); Kiran Kamtekar, Godmanchester (GB); Nazrul Islam, Godmanchester (GB)

(73) Assignee: Sumitomo Chemical Co., Ltd., Tokyo (JP)

(21) Appl. No.: 17/909,243

(22) PCT Filed: Mar. 2, 2021
(86) PCT No.: PCT/GB2021/050524

§ 371 Date: Sep. 2, 2022

(30) Foreign Application Priority Data
Mar. 3, 2020 (GB) 2003069.8

(51) Int. Cl. C09K 11/02; C09K 11/06; C12Q 1/6869; G01N 33/58

(52) U.S. Cl. CPC C09K 11/025; C09K 11/06; C12Q 1/6869; G01N 33/582; C09K 2211/1416; C09K 2211/1425

(57) ABSTRACT

A light-emitting particle comprising a core comprising a matrix material and a light-emitting system comprising a polymer and a shell layer comprising an inorganic oxide in contact with and surrounding the core.

### BACKGROUND

Embodiments of the present disclosure relate to light-emitting particles; methods of forming the same; and the use thereof as a luminescent marker.

### BACKGROUND

Light-emitting polymers have been disclosed as labelling or detection reagents.

Geng et al, “A general approach to prepare conjugated polymer dot embedded silica nanoparticles with a SiO2@CP@SiO2 structure for targeted HER2-positive cellular imaging”, Nanoscale, 2013, vol. 5, pp 8593-8601, describes silica-conjugated polymer (CP) nanoparticles having a “SiO₂@CP@SiO₂” structure.

Tansub et al, “Synthesis of Antibodies-Conjugated Fluorescent Dye-Doped Silica Nanoparticles for a Rapid Single Step Detection of Campylobacter jejuni in Live Poultry” Journal of Nanomaterials, Volume 2012, Article ID 865186 discloses silica nanoparticles containing a fluorescent dye of tris (2,2′-bipyridyl) dichlororuthenium(II) hexahydrate. Carboxyl modification of amine-functionalized nanoparticles with succinic anhydride is disclosed.

### SUMMARY

According to some embodiments, the present disclosure provides a light-emitting particle comprising a core comprising a matrix material and a light-emitting system comprising a polymer and a shell layer comprising an inorganic oxide in contact with and surrounding the core.

Optionally, the inorganic oxide is silica.

Optionally, the shell layer has a thickness of at least 2 nm.

Optionally, the shell layer has a thickness of no more than 100 nm.

Optionally, the polymer is a light-emitting polymer.

Optionally, the polymer has a solubility in water or a C₁₋₈ alcohol at 20° C. of at least 0.01 mg/ml.

Optionally, the matrix material is silica.

Optionally, the particle is a nanoparticle having a diameter of less than 1000 nm.

Optionally, a surface group is bound to the shell layer.

Optionally, the surface group comprises a carboxylic acid group or a salt or ester thereof.

Optionally, the surface group comprises a polyether chain.

According to some embodiments, the present disclosure provides a powder comprising light-emitting particles as described herein.

According to some embodiments, the present disclosure provides a dispersion comprising light-emitting particles as described herein dispersed in a liquid.

Optionally, the liquid is a buffer solution.

According to some embodiments, the present disclosure provides a method of forming light-emitting particles as described herein wherein formation of the core comprises polymerisation of a monomer for forming the matrix in the presence of the polymer.

Optionally, the polymer is in dissolved form.

Optionally, the shell comprises silica and formation of the shell comprise polymerisation of a monomer for forming silica in the presence of the core.

According to some embodiments, the present disclosure provides a method of marking a biomolecule, the method comprising the step of binding the biomolecule to a light-emitting marker particle as described herein.

According to some embodiments, the present disclosure provides an assay method for a target analyte comprising contacting a sample with light-emitting marker particles as described herein and determining any binding of the target analyte to the light-emitting marker.

In some embodiments, the sample contacted with the light-emitting marker particles is analysed by flow cytometry. Optionally, an amount of target analyte bound to the light-emitting marker particles is determined. Optionally, the sample comprises a mixture of cells and one or more different types of target cells bound to the light-emitting marker are identified and/or quantified.

In some embodiments, the target analyte bound to the light-emitting particles is separated from the target analyte which is not bound to the light-emitting particles.

In some embodiments, the present disclosure provides a method of sequencing nucleic acids comprising:

contacting a primed template nucleic acid molecule with a polymerase and a test nucleotide;
incorporating the test nucleotide into a primed strand of the primed template only if it comprises a base complementary to the next base of the template strand;
irradiating the primed strand; and
determining from luminance of the primed strand if the test nucleotide has been incorporated into the primed strand,
wherein the test nucleotide of the irradiated primed strand is bound to a light-emitting particle as described herein.

In some embodiments, the test nucleotide contacted with the polymerase and nucleic acid molecule is bound to the light-emitting particle.

In some embodiments, the light-emitting marker binds to the test nucleotide after incorporation of the test nucleotide into the primed strand.

### DESCRIPTION OF THE DRAWINGS

The disclosed technology and accompanying figures describe some implementations of the disclosed technology.

FIG. 1 is a schematic illustration of a particle according to some embodiments;

FIG. 2 is a schematic illustration of a method of forming a particle according to some embodiments; and

FIG. 3 is a graph of particle diameter vs. TEOS shelling agent added to a particle core.

The drawings are not drawn to scale and have various viewpoints and perspectives. The drawings are some implementations and examples. Additionally, some components and/or operations may be separated into different blocks or combined into a single block for the purposes of discussion of some of the embodiments of the disclosed technology. Moreover, while the technology is amenable to various modifications and alternative forms, specific embodiments have been shown by way of example in the drawings and are described in detail below. The intention, however, is not to limit the technology to the particular implementations described. On the contrary, the technology is intended to cover all modifications, equivalents, and alternatives falling within the scope of the technology as defined by the appended claims.

### DETAILED DESCRIPTION

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof means any connection or coupling, either direct or indirect, between two or more elements; the coupling or connection between the elements can be physical, logical, electromagnetic, or a combination thereof. Additionally, the words “herein,” “above,” “below,” and words of similar import, when used in this application, refer to this application as a whole and not to any particular portions of this application. Where the context permits, words in the Detailed Description using the singular or plural number may also include the plural or singular number respectively. The word “or,” in reference to a list of two or more items, covers all of the following interpretations of the word: any of the items in the list, all of the items in the list, and any combination of the items in the list. References to an atom include any isotope of that atom unless stated otherwise.

The teachings of the technology provided herein can be applied to other systems, not necessarily the system described below. The elements and acts of the various examples described below can be combined to provide further implementations of the technology. Some alternative implementations of the technology may include not only additional elements to those implementations noted below, but also may include fewer elements.

These and other changes can be made to the technology in light of the following detailed description. While the description describes certain examples of the technology, and describes the best mode contemplated, no matter how detailed the description appears, the technology can be practiced in many ways. Details of the system may vary considerably in its specific implementation, while still being encompassed by the technology disclosed herein. As noted above, particular terminology used when describing certain features or aspects of the technology should not be taken to imply that the terminology is being redefined herein to be restricted to any specific characteristics, features, or aspects of the technology with which that terminology is associated. In general, the terms used in the following claims should not be construed to limit the technology to the specific examples disclosed in the specification, unless the Detailed Description section explicitly defines such terms. Accordingly, the actual scope of the technology encompasses not only the disclosed examples, but also all equivalent ways of practicing or implementing the technology under the claims.

To reduce the number of claims, certain aspects of the technology are presented below in certain claim forms, but the applicant contemplates the various aspects of the technology in any number of claim forms.

In the following description, for the purposes of explanation, numerous specific details are set forth in order to provide a thorough understanding of implementations of the disclosed technology. It will be apparent, however, to one skilled in the art that embodiments of the disclosed technology may be practiced without some of these specific details.

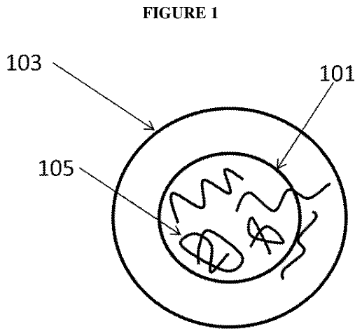

FIG. 1 schematically illustrates a light-emitting particle according to some embodiments of the present disclosure.

The light-emitting particle has a composite core 101 containing a matrix material and a light-emitting system including a polymer 105. The polymer is distributed within the composite core. Polymer chains may extend across some or all of the thickness of the core. Polymer chains may be contained within the core or may protrude through the surface of the core. In some embodiments, one or more polymer chains may be disposed on a surface of the core.

In some embodiments, the polymer 105 is a light-emitting polymer of the light-emitting system which, in use, emits light upon irradiation with an absorption wavelength of the polymer. According to these embodiments, the light-emitting system may comprise or consist of the light-emitting polymer.

In some embodiments, the polymer 105 is a host polymer of the light-emitting system, the light-emitting system further comprising a light-emitting material capable of receiving energy from the polymer. In these embodiments, in use there may be little or no emission from the host polymer.

The matrix material may be an inorganic oxide, optionally silica, iron oxide or alumina, preferably silica.

The present inventors have found that a light-emitting particle having a polymer in the core (e.g. a light-emitting polymer or a polymeric host for a separate light-emitting material) may have a tendency to aggregate and/or may have a surface that is difficult to functionalise. This effect may be particularly pronounced if the polymer has an affinity for the surface of the particle core, for example a hydrophilic polymer at the surface of a hydrophilic silica particle core, resulting in binding, e.g. van der Waals bonds, between the particle core surface and the polymer. The present inventors have found that providing a shell surrounding the polymer may overcome such problems.

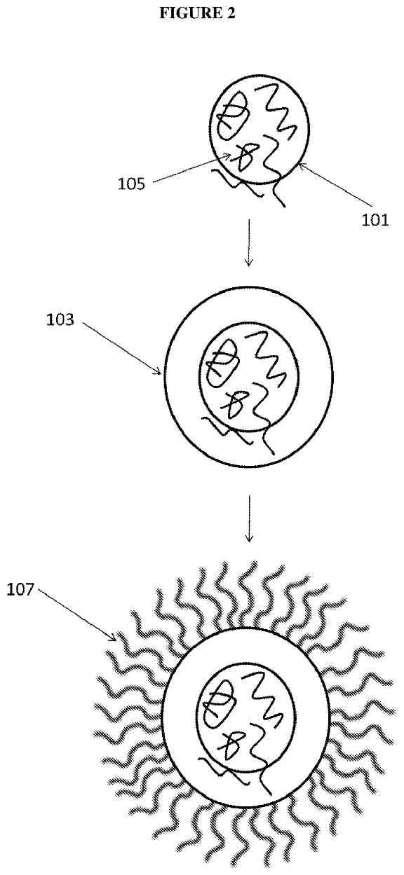

FIG. 2 illustrates a method of forming a light-emitting particle according to some embodiments.

The composite core 101 containing the matrix material having the light-emitting polymer distributed therein may be formed by any suitable method. In the case of a silica matrix material, a monomer for forming the silica may be polymerised in the presence of the polymer. Preferably, the silica is polymerised in the presence of dissolved polymer.

The core may be encapsulated with a shell 103 by any method known to the skilled person. In the case of a silica shell, a monomer for forming the silica may be polymerised in the presence of the core 101.

Upon formation of the shell, 103 any polymer chains 105 disposed on the surface of the core 101 or any polymer chains protruding from a surface of the core may be covered by the shell.

In some embodiments, following formation of the shell, the surface of the shell may be treated to provide surface groups 107, e.g. to provide functional groups at the shell surface or to enhance dispersion of the particles in a solvent, e.g. water. The functional groups at the shell surface may be capable of binding to a biomolecule group.

The matrix:organic light-emitting system weight ratio of the composite core may be in the range of 99:1-50:50, preferably 99:1-90:10.

The polymer may be uniformly distributed throughout the thickness of the composite core layer.

In some embodiments, at least some of the polymer in the composite core is covalently or non-covalently bound to the matrix.

In some embodiments, none of the light-emitting polymer chains are bound to the matrix.

The individual light-emitting polymer chains of the polymer may each independently have any configuration within the composite core including, without limitation, a folded or unfolded configuration. The configuration of the light-emitting polymer chains may be affected by the composite core formation process and conditions.

In some embodiments, there is no light-emitting material present in the shell.

Optionally, the particles are nanoparticles.

Preferably, the particles have a number average diameter of no more than 60 microns.

Preferably, the particles have a number average diameter of at least 10 nm.

Preferably, the cores have a number average diameter of no more than 50 microns.

Preferably, the cores have a number average diameter of at least 2 nm.

Preferably, the shells have an average thickness of no more than 1 micron. Preferably, the shells have a thickness of at least 2 nm. The particle brightness may be controlled, at least in in part, by selection of shell thickness.

Number average diameters provided herein are as measured by a Malvern Zetasizer Nano ZS.

Average shell thickness as provided herein is given by (number average particle diameter−number average core diameter)/2.

Particles as described herein may be provided as a powder. In some embodiments, the particles may be stored in a dry, optionally lyophilised, form. The particles may be stored in a frozen form.

Particles as described herein may be provided in a colloidal suspension comprising the particles suspended in a liquid. Preferably, the liquid is selected from water, C₁₋₈ alcohols and mixtures thereof. Preferably, the particles form a uniform (non-aggregated) colloid in the liquid. The liquid may be a solution comprising salts dissolved therein, optionally a buffer solution.

Optionally, light-emitting particles as described herein emit light in the visible range of the electromagnetic spectrum when excited by an energy source, e.g. a light source.

Emission from the light-emitting particles may have a peak wavelength in the range of 350-1,000 nm. Emission in the visible range may comprise or consist of red, green or blue light or a mixture thereof.

A blue light-emitting particle may have a photoluminescence spectrum with a peak of no more than 500 nm, preferably in the range of 400-500 nm, optionally 400-490 nm.

A green light-emitting particle may have a photoluminescence spectrum with a peak of more than 500 nm up to 580 nm, optionally more than 500 nm up to 540 nm.

A red light-emitting particle may have a photoluminescence spectrum with a peak of no more than more than 580 nm up to 630 nm, optionally 585 nm up to 625 nm.

The photoluminescence spectrum of light-emitting particles as described herein may be as measured using an Ocean Optics 2000+ spectrometer.

Optionally, light-emission from the light-emitting particles is observed upon irradiation with a light source having a peak wavelength in the range of 220-1800 nm. UV/vis absorption spectra of light-emitting particles as described herein may be as measured using a Cary 5000 UV-vis-IR spectrometer.

The light-emitting polymer may have a Stokes shift in the range of 10-850 nm.

Polymer

The polymer of the light-emitting system as described herein may be a fluorescent or phosphorescent light-emitting polymer.

The polymer of the light-emitting system as described herein may be a host polymer, the light-emitting system further comprising a light-emitting material. Exemplary light-emitting materials include, without limitation, fluorescent or phosphorescent organic materials and quantum dots.

The polymer of the light-emitting system may have a solubility in water or a C₁₋₈ alcohol at 20° C. of at least 0.01 mg/ml, optionally at least 0.1, 1, 5 or 10 mg/ml. Optionally, solubility is in the range of 0.01-10 mg/ml.

The polymer may have a solubility in a C₁₋₄ alcohol, preferably methanol, at 20° C. of at least 0.01 mg/ml, optionally at least 0.1, 1, 5 or 10 mg/ml.

Solubility may be as determined by visual observation under white and/or UV light after heating of a mixture of the solvent and the light-emitting polymer on a hotplate at 60° C. for 30 minutes with stirring and allowing the solution to cool to 20° C.

The polystyrene-equivalent number-average molecular weight (Mn) measured by gel permeation chromatography of polymers described herein may be in the range of about 1×10³ to 1×10⁸, and preferably 1×10⁴ to 5×10⁶. The polystyrene-equivalent weight-average molecular weight (Mw) of the light-emitting polymers described herein may be 1×10³ to 1×10⁸, and preferably 1×10⁴ to 1×10⁷.

A polymer as described herein may be a conjugated or non-conjugated polymer. By “conjugated polymer” as used herein is meant that a backbone of the polymer contains aromatic, heteroaromatic or vinylene groups which are directly conjugated to aromatic, heteroaromatic or vinylene groups of adjacent repeat units. The backbone may be conjugated along its entire length. The backbone may contain a plurality of conjugated sections which are not conjugated to one another.

A conjugated polymer as described herein may contain one or more of an arylene repeat unit; a heteroarylene repeat unit; and an arylamine repeat unit, each of which may be unsubstituted or substituted with one or more substituents.

Substituents may be selected from non-polar substituents, for example C₁₋₃₀ hydrocarbyl substituents; and polar substituents. Polar substituents may be ionic or non-ionic. A polar substituent may confer on the light-emitting polymer a solubility in a C₁₋₈ alcohol at 20° C. of at least 0.1 mg/ml, optionally at least 0.2, 03 or 0.5 mg/ml.

Non-polar substituents include, without limitation, C₁₋₃₀ hydrocarbyl substituents, e.g. C₁₋₂₀ alkyl, unsubstituted phenyl and phenyl substituted with one or more C₁₋₂₀ alkyl groups.

An exemplary non-ionic polar group has formula —O(R³O)_{q}-R⁴ wherein R³ in each occurrence is a C₁₋₁₀ alkylene group, optionally a C₁₋₅ alkylene group, wherein one or more non-adjacent, non-terminal C atoms of the alkylene group may be replaced with 0, R⁴ is H or C₁₋₅ alkyl, and q is at least 1, optionally 1-10. Preferably, q is at least 2. More preferably, q is 2 to 5. The value of q may be the same in all the polar groups of formula —O(R³O)_{q}-R⁴. The value of q may differ between non-ionic polar groups of the same polymer.

By “C₁₋₅ alkylene group” as used herein with respect to R³ is meant a group of formula —(CH₂)_{f}— wherein f is from 1-5.

Optionally, the polymer comprises non-ionic polar groups of formula —O(CH₂CH₂O)_{q}—R⁴ wherein q is at least 1, optionally 1-10 and R⁴ is a C₁₋₅ alkyl group, preferably methyl. Preferably, q is at least 2. More preferably, q is 2 to 5, most preferably q is 3.

Ionic substituents may be anionic or cationic.

Exemplary anionic groups are —COO⁻, a sulfonate group; hydroxide; sulfate; phosphate; phosphinate; or phosphonate. The counter cation of an anionic group may be selected from a metal cation, optionally Li⁺, Na⁺, K⁺, Cs⁺, preferably Cs⁺, and an organic cation, optionally ammonium, such as tetraalkylammonium, ethylmethyl imidazolium or pyridinium.

An exemplary cationic group is —N(R⁵)₃⁺ wherein R⁵ in each occurrence is H or C₁₋₁₂ hydrocarbyl. Preferably, each R⁵ is a C₁₋₁₂ hydrocarbyl, e.g. C₁₋₁₂ alkyl, unsubstituted phenyl and phenyl substituted with one or more C₁₋₆ alkyl groups. The counter anion of a cationic group may be a halide; a sulfonate group, optionally mesylate or tosylate; hydroxide; carboxylate; sulfate; phosphate; phosphinate; phosphonate; or borate.

A polar substituent may have formula —Sp-(R¹)n wherein Sp is a spacer group; n is at least 1, optionally 1, 2, 3 or 4; and; R¹ in each occurrence is independently an ionic or non-ionic polar group.

Preferably, Sp is selected from:
C₁₋₂₀ alkylene or phenylene-C₁₋₂₀ alkylene wherein one or more non-adjacent C atoms may be replaced with O, S, N or C═O;
a C₆₋₂₀ arylene or 5-20 membered heteroarylene, more preferably phenylene, which, other than the one or more substituents R¹, may be unsubstituted or substituted with one or more non-polar substituents, optionally one or more C₁₋₂₀ alkyl groups.
   “alkylene” as used herein means a branched or linear divalent alkyl chain.

Optionally, the conjugated light-emitting polymer contains one or more arylene repeat units selected from C₆₋₂₀ arylene repeat units, e.g. phenylene fluorene, indenofluorene, benzofluorene, dihydrophenanthrene, phenanthrene, naphthalene and anthracene repeat units.

Optionally, the polymer contains one or more arylene repeat units selected from formulae (III)-(VI):

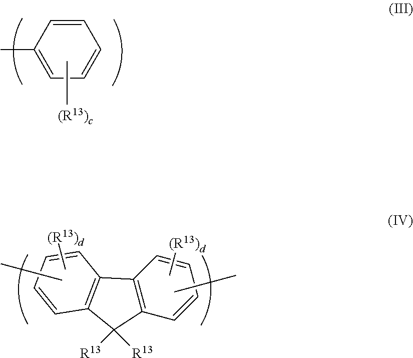

wherein R¹³ in each occurrence is independently a substituent and two R¹³ groups may be linked to form a ring; c is 0, 1, 2, 3 or 4, preferably 1 or 2; each d is independently 0, 1, 2 or 3, preferably 0 or 1; and e is 0, 1 or 2, preferably 2.

Each R¹³ group, where present, may be selected from a non-polar or polar substituent as described herein.

In some embodiments, two R¹³ groups may be linked to form a 6-membered ring or 7-membered ring. Optionally, two R¹³ groups are linked to form a ring in which the linked R¹³ groups form a C₄- or C₅- alkylene chain wherein one or more non-adjacent C atoms of the alkylene chain may be replaced with O, S, NR¹⁰ or Si(R¹⁰)₂ wherein R¹⁰ in each occurrence is independently a C₁₋₂₀ hydrocarbyl group.

An exemplary repeat unit in which two R¹³ groups are linked has formula (IVb):

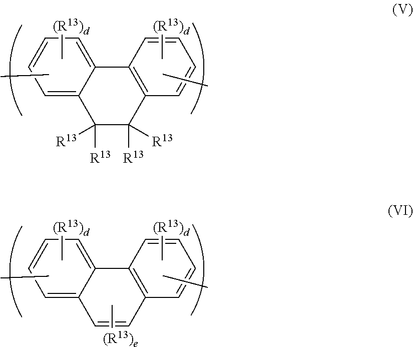

wherein each R¹² is independently H or R¹³, preferably H.

In some embodiments, no R¹³ groups are linked to one another.

A preferred arylene repeat unit has formula (IVa):

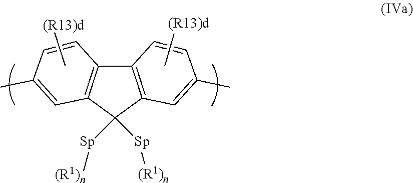

An exemplary repeat unit of formula (IVa) is:

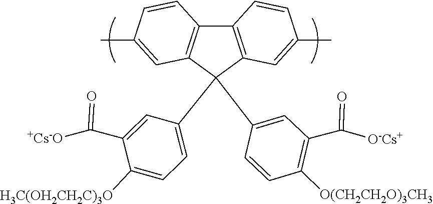

Repeat units comprising or consisting of one or more unsubstituted or substituted 5-20 membered heteroarylene groups in the polymer backbone include, without limitation, thiophene repeat units, bithiophene repeat units, benzothiadiazole repeat units, and combinations thereof. Exemplary heteroarylene repeat units include repeat units of formulae (VIII)-(XI):

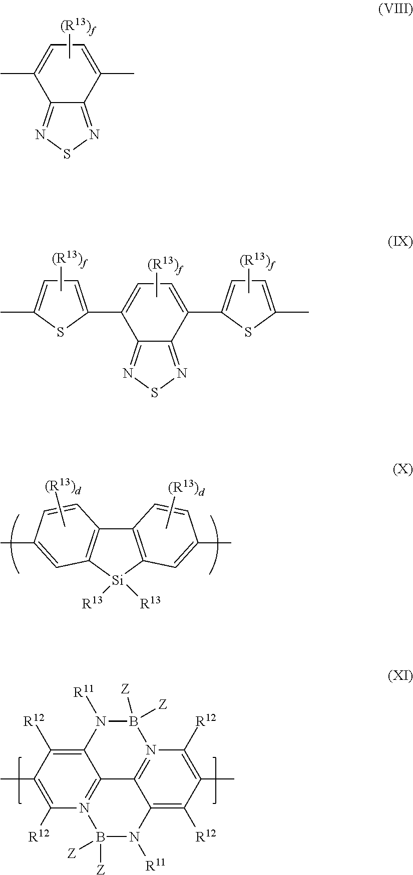

wherein R¹¹ independently in each occurrence is a C₁₋₂₀ hydrocarbyl group; Z in each occurrence is independently a substituent, preferably F or a C₁₋₂₀ hydrocarbyl group; f is 0, 1 or 2 and R¹², R¹³ and d are as described above.

A C₁₋₂₀ hydrocarbyl group as described anywhere herein is optionally selected from C₁₋₂₀ alkyl, unsubstituted phenyl and phenyl substituted with one or more C₁₋₁₂ alkyl groups.

Each R¹³ may independently be selected from polar and non-polar substituents as described above.

Arylamine repeat units may have formula (XII):

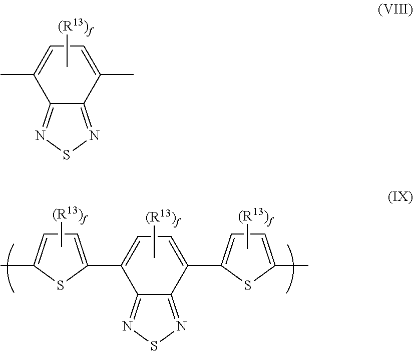

wherein Ar⁸, Ar⁹ and Ar¹⁰ in each occurrence are independently selected from substituted or unsubstituted aryl or heteroaryl, g is 0, 1 or 2, preferably 0 or 1, R¹³ independently in each occurrence is a substituent, and x, y and z are each independently 1, 2 or 3.

R⁹, which may be the same or different in each occurrence when g is 1 or 2, is preferably selected from the group consisting of alkyl, optionally C₁₋₂₀ alkyl, Ar¹¹ and a branched or linear chain of Ar¹¹ groups wherein Ar¹¹ in each occurrence is independently substituted or unsubstituted aryl or heteroaryl.

Any two aromatic or heteroaromatic groups selected from Ar⁸, Ar⁹, and, if present, Ar¹⁰ and Ar¹¹ that are directly bound to the same N atom may be linked by a direct bond or a divalent linking atom or group. Preferred divalent linking atoms and groups include O, S; substituted N; and substituted C.

Ar⁸ and Ar¹⁰ are preferably C₆₋₂₀ aryl, more preferably phenyl, which may be unsubstituted or substituted with one or more substituents.

In the case where g=0, Ar⁹ is preferably C₆₋₂₀ aryl, more preferably phenyl, that may be unsubstituted or substituted with one or more substituents.

In the case where g=1, Ar⁹ is preferably C₆₋₂₀ aryl, more preferably phenyl or a polycyclic aromatic group, for example naphthalene, perylene, anthracene or fluorene, which may be unsubstituted or substituted with one or more substituents. It is particularly preferred that Ar⁹ is anthracene when g=1.

R⁹ is preferably Ar¹¹ or a branched or linear chain of Ar¹¹ groups. Ar¹¹ in each occurrence is preferably phenyl that may be unsubstituted or substituted with one or more substituents.

Exemplary groups R⁹ include the following, each of which may be unsubstituted or substituted with one or more substituents, and wherein * represents a point of attachment to N:

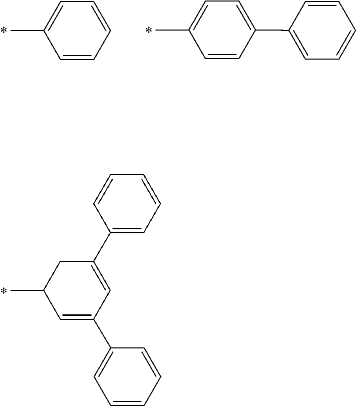

x, y and z are preferably each 1.

Ar⁸, Ar⁹, and, if present, Ar¹⁰ and Ar¹¹ are each independently unsubstituted or substituted with one or more, optionally 1, 2, 3 or 4, substituents.

Substituents may independently be selected from non-polar or polar substituents as described herein.

Preferred substituents of Ar⁸, Ar⁹, and, if present, Ar¹⁰ and Ar¹¹ are C₁₋₄₀ hydrocarbyl, preferably C₁₋₂₀ alkyl.

Preferred repeat units of formula (XII) include unsubstituted or substituted units of formulae (XII-1), (XII-2) and (XII-3):

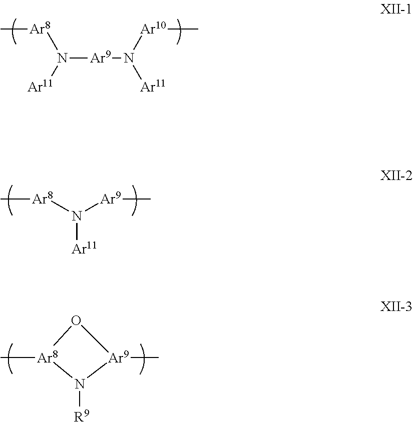

Conjugated polymers as described herein may be formed by polymerising monomers comprising leaving groups that leave upon polymerisation of the monomers to form conjugated repeat units. Exemplary polymerization methods include, without limitation, Yamamoto polymerization as described in, for example, T. Yamamoto, “Electrically Conducting And Thermally Stable pi-Conjugated Poly(arylene)s Prepared by Organometallic Processes”, Progress in Polymer Science 1993, 17, 1153-1205, the contents of which are incorporated herein by reference and Suzuki polymerization as described in, for example, WO 00/53656, WO 2003/035796, and U.S. Pat. No. 5,777,070, the contents of which are incorporated herein by reference.

The polystyrene-equivalent number-average molecular weight (Mn) measured by gel permeation chromatography of the polymers described herein may be in the range of about 1×10³ to 1×10⁸, and preferably 1×10⁴ to 5×10⁶. The polystyrene-equivalent weight-average molecular weight (Mw) of the polymers described herein may be 1×10³ to 1×10⁸, and preferably 1×10⁴ to 1×10⁷.

Composite Core Formation

In the case of a silica matrix, the composite core may be formed by reacting a monomer for forming silica in the presence of dissolved polymer(s) to be incorporated into the composite core in the presence of the polymer.

Optionally, the silica monomer is an alkoxysilane, preferably a trialkoxy or tetra-alkoxysilane, optionally a C₁₋₁₂ trialkoxy or tetra-alkoxysilane, for example tetraethyl orthosilicate. The silica monomer may be substituted only with alkoxy groups or may be substituted with one or more groups.

Optionally, the silica monomer is polymerised in a liquid comprising or consisting of an ionic solvent or a protic solvent, preferably a solvent selected from water, alcohols and mixtures thereof. Exemplary alcohols include, without limitation, methanol, ethanol, 1-propanol, isopropanol, 1-butanol, 2-butanol, t-butanol, pentanol, hexanol, heptanol, octanol and mixtures thereof. Preferably, the solvent system does not comprise a non-alcoholic solvent other than water. Preferably, the polymer is dissolved in the liquid.

Polymerisation may be carried out in the presence of a base, e.g. a metal hydroxide, preferably alkali metal hydroxide, ammonium hydroxide or tetraalkylammonium hydroxide.

In the case of a silica shell, formation of the shell may be carried out by polymerisation of a silica monomer which may be as described with reference to the core, in the presence of the core.

Preferably, the shell is formed in the presence of the core and in the absence of any free polymer of the light-emitting system, i.e. polymer which is not part of the core.

In some embodiments, a core comprising a silica matrix is formed in a first step and a shell comprising silica is formed in a second step without first isolating the core from the reaction mixture in which it was formed. Preferably, no polymer of the light-emitting system remains in solution following the first step. This may be verified by, for example, testing the solution separated from the core formed in the first step for residual fluorescence.

The particles may be isolated following formation of the shell and resuspended in an aqueous solvent, an organic solvent or a mixture thereof. The composite particles may be isolated from the reaction mixture by centrifuging.

Surface Groups

One or more surface groups may be bound, e.g. covalently bound, to the outer surface of the shell.

In some embodiments, a functional group is bound to the shell. The functional group may be bound directly to the surface of shell or bound through a surface binding group.

In some embodiments, the functional group comprises a carboxylic acid group or salt, ester, or acid chloride thereof. The functional carboxylic acid group may be reacted with a wide range of groups including, without limitation, alcohols, e.g. a glycol such as polyethyleneglycol, and azides.

In some embodiments, the acid functional group may be used to bind a biomolecule to the surface of the particle, e.g. biotin, which may in turn be used to bind to a target biomolecule in an assay.

In some embodiments, the acid group may be used in an assay to bind directly to a target biomolecule, e.g. an amino group of a target antibody, protein or oligonucleotide.

The surface of the shell may carry reactive groups, e.g. hydroxyl groups which may be reacted to bind a functional group to the particle.

In some embodiments, a reactive material is reacted directly with the reactive surface groups.

In some embodiments, the reactive surface groups are modified before the functional group is bound thereto. In some embodiments, amino groups are formed at the surface of the particle. Optionally hydroxyl reactive surface groups may be reacted with an aminosilane, e.g. a compound of formula (I):

(R¹O)₃Si—R²—N(R³)₂ (I)

wherein R¹ in each occurrence is a C₁₋₂₀ hydrocarbyl group, preferably a C₁₋₁₂ alkyl group; R² is a C₁₋₂₀ hydrocarbylene group, preferably a C₁₋₂₀ alkylene group; and R³ in each occurrence is H or a C₁₋₂₀ hydrocarbyl group, preferably H.

Amino reactive groups at the surface of the particle may be reacted with a reactive material for forming the functional group.

The reactive material may contain the functional group or a derivative thereof and a reactive group for reacting with a reactive surface group.

The reactive material may contain a carboxyl group or a derivative thereof, e.g. an acid chloride, acid anhydride or ester including, without limitation NHS esters, for reaction with a reactive surface group.

In some embodiments, the reactive material has formula (II):

HOOC—(C₂H₅O)n-COOH (II)

wherein n is at least 1, optionally 1-100.

In some embodiments, the reactive material is an anhydride (e.g. succinic anhydride) which, upon reaction with the particle surface, forms a first carboxylic acid derivative bound to the particle surface, and a second free carboxylic acid functional group.

The light-emitting particle may comprise two or more different surface groups. Optionally, one surface group comprises a functional group and another surface group does not comprise the functional group. The, or each, surface group may comprise a polyether chain. By “polyether chain” as used herein is meant a divalent chain comprising a plurality of ether groups, e.g. a polyethylene glycol chain.

Applications

Light-emitting particles as described herein may be used as luminescent probes in an immunoassay such as a lateral flow or solid state immunoassay. Optionally the light emitting particles are for use in fluorescence microscopy, flow cytometry, nucleic acid sequencing methods for example next generation sequencing, in-vivo imaging, or any other application where a light-emitting marker configured to bind to a target analyte is brought into contact with a sample to be analysed. The applications can medical, veterinary, agricultural or environmental applications whether involving patients (where applicable) or for research purposes.

In use as light-emitting marker particles, the light-emitting particles may be irradiated at an absorption wavelength of the light-emitting polymer or, if present, an absorption wavelength of a material configured to transfer energy to the light-emitting polymer, and emission from the light-emitting polymer may be detected. In some embodiments, the sample following contact with the particles is analysed by flow cytometry. In flow cytometry, the particles are irradiated by at least one wavelength of light, optionally two or more different wavelengths, e.g. one or more wavelengths including at least one of 355, 405, 488, 530, 562 and 640 nm±10 nm. Light emitted by the particles may be collected by one or more detectors. Detectors may be selected from, without limitation, photomultiplier tubes and photodiodes. To provide a background signal for calculation of a staining index, measurement may be made of particles mixed with cells which do not bind to the particles.

In some embodiments, a target analyte may be immobilised on a surface carrying a group capable of binding to the target analyte, either before or after the target analyte binds to the particles. The particles bound to the target analyte immobilised on the surface may then be separated from any light-emitting particles which are not bound to the target analyte.

The light-emitting particles may be configured to bind to a target biomolecule including without limitation DNA, RNA, peptides, carbohydrates, antibodies, antigens, enzymes, proteins and hormones. In some embodiments, the target biomolecule is a biomolecule (e.g. a protein) at a surface of a cell.

It will be understood that biomolecule binding groups may be provided on the surface of the particle according to the target biomolecule.

In some preferred embodiments, the particle comprises biotin which binds directly to a target analyte.

In some embodiments, a first biotin group is bound to the particle which, in turn, is bound to a protein having a plurality of biotin binding sites, preferably streptavidin, neutravidin, avidin or a recombinant variant or derivative thereof and a biotinylated biomolecule having a second biotin group is bound to the same protein. The biotinylated biomolecule may be selected according to the target analyte. The biotinylated biomolecule may comprise an antigen binding fragment, e.g. an antibody, which may be selected according to a target antigen.

In the case where the light-emitting particles are used in a nucleic acid sequencing method, he surface of the light-emitting particle may carry a group capable of binding to a nucleotide to form a test nucleotide. For example, one of the test nucleotide and the light-emitting particle may be functionalised with biotin and the other of the test nucleotide and the conjugated polymer may be functionalised with avidin, streptavidin, neutravidin or a recombinant variant thereof.

### EXAMPLES

### Example 1 Synthesis of Unshelled Silica-LEP Nanoparticle

Light-Emitting Polymer 1 (LEP1) was dissolved in anhydrous methanol at a concentration of 1 mg/mL by heating the solution to 60° C. for 15 minutes and then cooling to room temperature. To 0.5 mL of this LEP solution in a 10 mL amber vial was added 0.83 mL of anhydrous methanol, 0.66 mL of octanol and 0.13 mL ammonium hydroxide (28-30% aq.). The vial was closed with a tightly fitted cap with septum and the reaction mixture was heated to 60° C. with stirring. After 5 minutes, a solution of tetraethyl orthosilicate (TEOS, 0.025 mL), methanol (0.33 mL) and octanol (0.17 mL) was rapidly injected into the reaction mixture and stirring at 60° C. was continued for a further 1 h. After cooling to room temperature, the reaction mixture was transferred to 2×1.5 mL microcentrifuge tubes and centrifuged at 14,000 rpm for 4 minutes and the supernatant was decanted off. The pelleted nanoparticles were redispersed in methanol (total volume=2.5 mL) by closing the microcentrifuge tubes and immersing in a water bath with fitted an ultrasonic horn (amplitude 20%, 5×5s pulses). Washing was repeated ×2 by further centrifugation, decantation and ultrasonication using 2.5 mL of fresh methanol for each wash. The nanoparticles were resuspended in 2.5 mL methanol for storage and their size was measured by DLS using a Malvern Zetasizer Nano S.

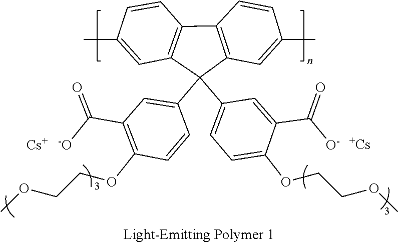

### Example 2 Silica-Shelling of Silica-LEP Nanoparticle

1 mL of the nanoparticle suspension formed by the method described in Example 1 was transferred to a 1.5 mL microcentrifuge tube and centrifuged at 14000 rpm for 4 minutes to isolate the solids and the supernatant was removed by decantation. The pelleted nanoparticles were then resuspended in 1 mL of a 2:1 mixture of methanol and octanol by immersing in a water bath with fitted an ultrasonic horn (amplitude 20%, 5×5s pulses). Ammonium hydroxide (0.075 mL, 28-30% aq) was mixed with this reaction mixture, followed by 0.003 mL or 0.005 mL of TEOS. The dispersion was then mixed at room temperature by leaving on rollers for 1 hour, followed by washing ×3 by centrifugation, decantation and resuspension in 1 mL methanol as described in example 1. The nanoparticles were finally resuspended in 1 mL of methanol for DLS analysis and storage.

With reference to Table 1, Addition of TEOS to the particles shows an increase in Z-average and number average (N average) diameter, as measured by DLS in methanol. The polydispersity (PdI) remains very low (<0.1) after the shelling reaction.

TABLE 1

| | | +0.003 mL | +0.005 mL |
|---|---|---|---|
| | Unshelled | TEOS | TEOS |
| Z average/nm | 121 | 136.1 | 149.4 |
| PdI | 0.009 | 0.025 | 0.056 |
| N average/nm | 102.0 | 116.8 | 120.3 |

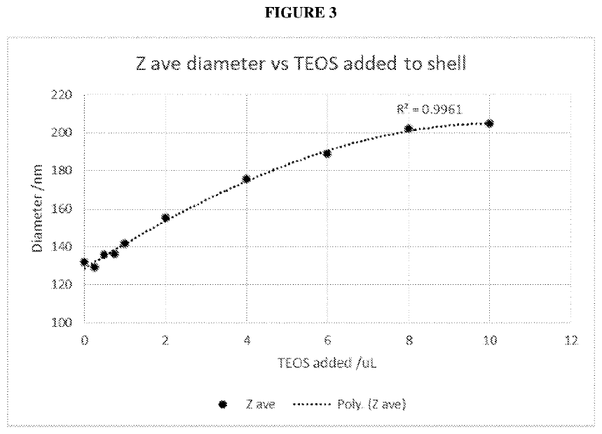

The effect of the amount of TEOS on shell thickness and, therefore, particle diameter was studied across a wider range of TEOS amounts. The results set out in FIG. 3 illustrate a clear relationship between amount of TEOS and particle diameter.

### Example 3 Amino Surface Modification of Silica-LEP Nanoparticles

Unshelled silica-LEP nanoparticles (as formed using the method from Example 1) or shelled silica-LEP nanoparticles (as formed using the method from Example 2) were further modified as follows. To 1 mL of the nanoparticle dispersion in methanol was added (3-aminopropyl)triethoxysilane (0.04 mL). The dispersion was then mixed at room temperature by leaving on rollers for 1 hour, followed by washing ×3 by centrifugation, decantation and resuspension in 1 mL methanol as described in example 1. The aminated nanoparticles were finally resuspended in 1 mL of methanol for storage.

### Example 4 Carboxy-Functionalisation of Aminated Silica-LEP Nanoparticles by Reaction with Succinic Anhydride

Aminated nanoparticles in methanol (1 mL), as formed in Example 3, were transferred to a 1.5 mL microcentrifuge tube and centrifuged for 4 minutes at 14,000 rpm and the supernatant was removed by decantation. To the pelleted nanoparticles was added 1 mL of a 5 wt. % solution of succinic anhydride in dimethyl formamide and the particles were resuspended in this solution by immersing in a water bath with fitted an ultrasonic horn (amplitude 20%, 5×5s pulses). The dispersion was then mixed at room temperature by leaving on rollers for 1 hour, followed by washing ×3 by centrifugation, decantation and resuspension in 1 mL methanol as described in example 1. The aminated nanoparticles were finally resuspended in 1 mL of methanol for storage.

### Example 5 Stability of Carboxy-Functionalised Silica-LEP Nanoparticles in Phosphate Buffered Saline

Solid content of carboxy-modified nanoparticles in methanol was determined by centrifuging a 0.2 mL aliquot (14,000 rpm, 4 min), decanting off the supernatant, leaving to dry in air for 16 hours and then weighing the residual solids. After this measurement, a volume of nanoparticles containing 1 mg solids was isolated by centrifuging and decanting the supernatant. The solid nanoparticles were then resuspended in 1 mL of phosphate buffered saline (pH 7.4) by immersing in a water bath with fitted an ultrasonic horn (amplitude 20%, 5×5s pulses). DLS measurements were taken immediately after resuspension and after the time intervals shown in Table 2.

TABLE 2

| Vol. TEOS | | | | | |
|---|---|---|---|---|---|
| in shelling | Z-average diameter, nm (and PdI) | | | | |
| step | | t = | t = | t = | t = |
| (mL) | t = 0 h | 5 h | 24 h | 48 h | 1,176 h |
| 0 | Poor data quality | n/a | n/a | n/a | n/a |
| | (aggregated) | | | | |
| 0.003 | 144 | 147 | 150 | 149 | 152 |
| | (0.002) | (0.07) | (0.08) | (0.08) | (0.05) |
| 0.005 | 151 | 154 | 156 | 153 | 156 |
| | (0.04) | (0.05) | (0.06) | (0.05) | (0.05) |

As shown in Table 2, the shelled nanoparticles containing a core of silica and LEP1 have much less tendency to aggregate than the unshelled nanoparticles.

For comparison, stability of these nanoparticles was compared with an unshelled nanoparticle having a core consisting of silica only. As shown in Table 3, such a nanoparticle shows significantly greater stability than an unshelled nanoparticle having a core of silica and LEP1. In Table 3, the stability period is the time taken for Z-average diameter as measured by dynamic light scattering to increase by 10%.

TABLE 3

| | Succinic anhydride product stability |
|---|---|
| Nanoparticle | period (1 × PBS, 1 mg/mL) |
| Silica core, unshelled | >38 days |
| Silica-LEP1 core, | n/a |
| unshelled | |
| Silica-LEP1 core + | >49 days |
| 0.003 mL TEOS | |
| Silica-LEP1 core + | >49 days |
| 0.005 mL TEOS | |

### Example 6 One-Pot Formation of Shelled Silica-LEP Nanoparticles

To a solution of LEP1 in anhydrous methanol (2.5 mL, formed as described in Example 1) was added anhydrous methanol (5.33 mL), octanol (4.17 mL) and ammonium hydroxide (28-30%, aq.). The solution was capped tightly in a 20 mL amber vial with septum and heated to 60° C. with stirring. After 5 minutes, a solution of TEOS (0.125 mL) in methanol (0.5 mL) was rapidly injected into the reaction mixture and stirring was continued for a further 1 h. After this time, the reaction mixture was cooled to room temperature and additional TEOS (19 μL) was added, followed by stirring at room temperature for 1 hour. The nanoparticles were then washed using the procedure described in Example 1. The final volume of methanol used for resuspending the nanoparticles for storage was 11 mL. For comparison, unshelled nanoparticles were made by the same process, but without the additional 19 μL of TEOS being added after 1 hour.

Amino modification and reaction with succinic anhydride were carried out as described in Examples 3 and 4. For comparison, unshelled nanoparticles were made by the same process, but without the additional 19 μL of TEOS being added after 1 hour. DLS data for the nanoparticles after each stage in the process is shown in Table 5.

TABLE 5

| | Z-average diameter, nm (and PdI) | | | |
|---|---|---|---|---|
| | After | COOH- | COOH- | COOH- |
| | formation, | modified, | modified, | modified, |
| | in | in | in PBS | in PBS (t = 35 |
| Nanoparticles | methanol | methanol | (t = 0) | days) |
| Unshelled | 112 | 118 | Poor data | n/a |
| | (0.07) | (0.13) | quality | |
| | | | (aggregated) | |
| Shelled | 109 | 108 | 106 | 100 |
| (1 pot method) | (0.06) | (0.04) | (0.02) | (0.01) |

1. A light-emitting particle comprising a composite core comprising a matrix material and a light-emitting system comprising a polymer wherein the polymer is distributed within the core; and a shell layer comprising an inorganic oxide in contact with and surrounding the core.

2. The light-emitting particle according to claim 1 wherein the inorganic oxide is silica.

3. The light-emitting particle according to claim 1 wherein the shell layer has a thickness of at least 2 nm.

4. The light-emitting particle according to claim 1 wherein the shell layer has a thickness of no more than 100 nm.

5. The light-emitting particle according to claim 1 wherein the polymer is a light-emitting polymer.

6. The light-emitting particle according to claim 1 wherein the polymer has a solubility in water or a C₁₋₈ alcohol at 20° C. of at least 0.01 mg/ml.

7. The light-emitting particle according to claim 1 wherein the matrix material is silica.

8. The light-emitting particle according to claim 1 wherein the particle is a nanoparticle having a diameter of less than 1000 nm.

9. The light-emitting particle according to claim 1 wherein a surface group is bound to the shell layer.

10. The light-emitting particle according to claim 9 wherein the surface group comprises a carboxylic acid group or a salt or ester thereof.

11. The light-emitting particle according to claim 9 wherein the surface group comprises a polyether chain.

12. A powder comprising light-emitting particles according to claim 1.

13. A dispersion comprising light-emitting particles according to claim 1 dispersed in a liquid.

14. The dispersion according to claim 13 wherein the liquid is a buffer solution.

15. A method of forming light-emitting particles according to claim 1 wherein formation of the core comprises polymerisation of a monomer for forming the matrix in the presence of the polymer.

16. The method according to claim 15 wherein the polymer is in dissolved form.

17. The method according to claim 15 or 16 wherein the shell comprises silica and wherein formation of the shell comprise polymerisation of a monomer for forming silica in the presence of the core.

18. A method of marking a biomolecule, the method comprising the step of binding the biomolecule to a light-emitting particle according to claim 1.

19. An assay method for a target analyte comprising contacting a sample with light-emitting particles according to claim 1 and determining any binding of the target analyte to the light-emitting marker.

20-23. (canceled)

24. A method of sequencing nucleic acids comprising:
contacting a primed template nucleic acid molecule with a polymerase and a test nucleotide;
incorporating the test nucleotide into a primed strand of the primed template only if it comprises a base complementary to the next base of the template strand;
irradiating the primed strand; and
determining from luminance of the primed strand if the test nucleotide has been incorporated into the primed strand,
wherein the test nucleotide of the irradiated primed strand is bound to a light-emitting particle according to claim 1.
